(19) United States
(12) Patent Application Publication
(10) Pub. No.: US 2008/0300477 A1
(43) Pub. Date: Dec. 4, 2008

(54) SYSTEM AND METHOD FOR CORRECTION OF AUTOMATED IMAGE REGISTRATION

(75) Inventors: Charles Frederick Lloyd, Reading, MA (US); Jon Thomas Lea, Hampstead, NH (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/755,118

(22) Filed: May 30, 2007

(51) Int. Cl. A61B 5/05

(52) U.S. Cl. 600/407

(57) ABSTRACT

An image guided surgical system and method for correction of automated image registration via user interaction. The system and method comprising at least one imaging apparatus adapted to acquire a first image and a second image of a region of interest of a subject, a registration component adapted to perform a registration of the second image to a dataset of the first image, at least one display for displaying a visualization of the registration of the second image to a dataset of the first image as it is occurring, and a user interface for manipulating the visualization of the registration to correct any misalignments between the first image and the second image in the registration.

### BACKGROUND OF THE INVENTION

This disclosure relates generally to image-guided surgery (or surgical navigation). In particular, this disclosure relates to a medical navigation system with a system and method for correcting and refining automated image based registration via user interaction.

Medical navigation systems track the precise location of surgical instruments and implants in relation to multidimensional images of a patient's anatomy. Additionally, medical navigation systems use visualization tools to provide the surgeon with co-registered views of these surgical instruments and implants with the patient's anatomy. The multidimensional images of a patient's anatomy may include computed tomography (CT) imaging data, magnetic resonance (MR) imaging data, positron emission tomography (PET) imaging data, ultrasound imaging data, X-ray imaging data, or any other suitable imaging data, as well as any combinations thereof. Medical navigation technology has been applied to a wide variety of medical procedures including cranial neurosurgeries; neurointerventions; ear, nose and throat (ENT) procedures; spinal surgeries; orthopedic surgeries; aortic stenting procedures, etc.

Several of these medical procedures require very precise planning for placement of surgical instruments and/or implants that are internal to the body or difficult to view during the procedure. For example, the placement of pedicle screws during spinal surgery require precise visualization of the entry points and the projected path of the instruments and implants through the pedicle bone to their desired position. These are best viewed on 3D images acquired during the procedure.

Registration of 3D image datasets (CT, MR, PET, ultrasound, etc.) to a known reference frame can be a difficult problem in the operating room. The initial registration is typically defined by identifying common fiducial points within a region of interest between a previously acquired 3D image dataset and a set of 2D or 3D fluoroscopic images acquired during the procedure. Image based registration algorithms can simplify the surgical workflow by using images that are available during the procedure without requiring direct contact with rigid patient landmarks.

A problem with image based registration algorithms is that they may not be able to accurately correct for certain alignment problems that are intuitive for an experienced technician or user to see and correct during the registration process. An example of an alignment problem would be a rotation of an image around the patient's axial direction.

Thus, it is highly desirable to provide an interactive image registration and refinement process to correct alignment problems during a procedure. Therefore, there is a need for a system and method for correcting automated image based registration via user interaction.

### BRIEF DESCRIPTION OF THE INVENTION

In an embodiment, a medical navigation system comprising at least one imaging apparatus adapted to acquire a first image and a second image of a region of interest of a subject, a registration component adapted to perform a registration of the second image to a dataset of the first image, at least one display for displaying a visualization of the registration of the second image to a dataset of the first image as it is occurring, and a user interface for manipulating the visualization of the registration to correct any misalignments between the first image and the second image in the registration.

In an embodiment, a method for performing image registration comprising acquiring a first image and a second image of a region of interest of a patient, performing a registration of the second image to a dataset of the first image, viewing a visualization of the registration on at least one display as the registration is occurring, and manipulating the visualization of the registration to correct any misalignments between the first image and the second image in the registration using a user interface.

In an embodiment, a computer-readable medium including a set of instructions for execution on a computer, the set of instructions comprising an acquisition routine for acquiring a first image and a second image of a region of interest of a patient, a registration routine for registering the second image to a dataset of the first image, a visualization routine for visualizing the registration on a display while the registration is proceeding, and a user interaction routine for manipulating the registration to correct any misalignments between the first image and the second image.

Various other features, objects, and advantages of the invention will be made apparent to those skilled in the art from the accompanying drawings and detailed description thereof.

### BRIEF DESCRIPTION OF THE DRAWINGS

FIG. 1 is an exemplary schematic diagram of an embodiment of a medical navigation system;

FIG. 2 is an exemplary block diagram of an embodiment of a medical navigation system;

FIG. 3 is an exemplary flow diagram of an embodiment of a method for performing image registration;

FIG. 4 is an exemplary flow diagram of an embodiment of a method for performing image registration;

FIG. 5A is an exemplary diagram of a misaligned first image and a second image during image registration; and

FIG. 5B is an exemplary diagram of an aligned first image and second image after user interaction to correct the misalignment in image registration as shown in FIG. 5A.

### DETAILED DESCRIPTION OF THE INVENTION

In surgical procedures, access to the body is obtained through one or more small percutaneous incisions or one larger incision in the body. Surgical instruments and/or implants are inserted through these openings and directed to a region of interest within the body. Direction of the surgical instruments or implants through the body is facilitated by navigation technology wherein the real-time location of a surgical instrument or implant is measured and virtually superimposed on an image of the region of interest. The image may be a pre-acquired image, or an image obtained in near real-time or real-time using known imaging technologies such as computed tomography (CT), magnetic resonance (MR), positron emission tomography (PET), ultrasound, X-ray, or any other suitable imaging technology, as well as any combinations thereof.

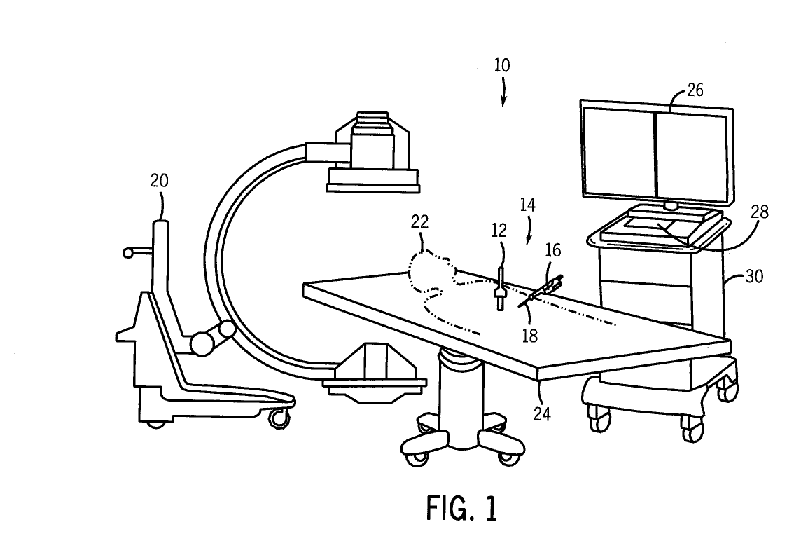

Referring now to FIG. 1, a medical navigation system (e.g., a surgical navigation system), designated generally by reference numeral 10 is illustrated. The system 10 includes at least one electromagnetic field generator 12 positioned proximate to a surgical field of interest 14; at least one electromagnetic sensor 16 attached to at least one navigated surgical instrument 18 to which an implant may be attached, the at least one electromagnetic sensor 16 communicating with and receiving data from the at least one electromagnetic field generator 12; a navigation apparatus 30 coupled to and receiving data from the at least one electromagnetic sensor 16 and the at least one electromagnetic field generator 12; at least one imaging apparatus 20 coupled to the navigation apparatus 30 for performing imaging on a patient 22 in the surgical field of interest 14, the system of FIG. 1 showing the patient 22 positioned on a table 24 during a surgical procedure; and at least one display 26 coupled to the navigation apparatus 30 for displaying imaging and tracking data from the medical navigation system. The system further includes a user interface 28 coupled to the navigation apparatus 30 for manipulating or correcting errors in the image registration process.

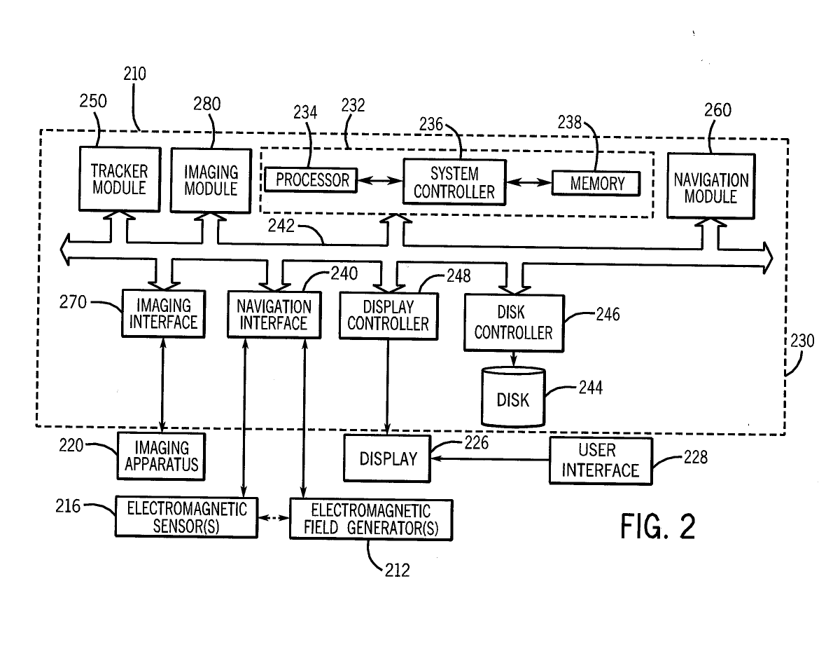

The navigation apparatus 30 may include at least one computer; at least one interface for communicating with the imaging apparatus 20, the at least one electromagnetic field generator 12, and the at least one electromagnetic sensor 16; a tracker module; a navigation module; an imaging module; and at least one storage device. A description of these components and there operation are described with reference to FIG. 2 below.

The display 26 is configured to show the image based registration process as it is progressing. The display 26 is also configured to show the real-time position and orientation of the at least one surgical instrument 18 or at least one implant attached to the tip or end of the at least one surgical instrument 18 on a registered image of the patient's anatomy. The graphical reference of the at least one surgical instrument 18 or at least one implant depicted on the display may appear as a line rendering, a few simply shaded geometric primitives, or a realistic 3D model from a computer-aided design (CAD) file.

The medical navigation system 10 is configured to operate with at least one electromagnetic field generator 12 and at least one electromagnetic sensor 16 to determine the position and orientation of the at least one device 18 or an implant. The at least one electromagnetic field generator 12 and the at least one electromagnetic sensor 16 may be coupled to a navigation interface on the navigation apparatus 30 through either a wired or wireless connection.

In an exemplary embodiment, the at least one electromagnetic field generator 12 may be an electromagnetic field transmitter. The electromagnetic field transmitter may be a transmitter coil array including at least one coil, at least one coil pair, at least one coil trio, or a coil array for generating an electromagnetic field in response to a current being applied to at least one coil. In an exemplary embodiment, the at least one electromagnetic sensor 16 may be an electromagnetic field receiver including at least one coil, at least one coil pair, at least one coil trio, or a coil array with electronics for digitizing magnetic field measurements detected by the electromagnetic field receiver. The electromagnetic field receiver detecting the electromagnetic field being generated by the electromagnetic field transmitter. It should, however, be appreciated that according to alternate embodiments the at least one electromagnetic field generator may be an electromagnetic sensor or an electromagnetic field receiver, and the at least one electromagnetic sensor may be an electromagnetic field generator.

In an exemplary embodiment, the at least one electromagnetic field generator 12 or an additional electromagnetic field generator may act as a dynamic reference that may be rigidly attached to the patient 22 in the surgical field of interest 14. This dynamic reference generates a different electromagnetic field (e.g., a different frequency) from the other electromagnetic field generators, and creates a local reference frame for the navigation system around the patient's anatomy in the surgical field of interest. Typically, the dynamic reference used by a navigation system is registered to the patient's anatomy prior to surgical navigation. Registration of the reference frame impacts the accuracy of a navigated instrument in relation to a displayed image.

The system 10 enables a surgeon to continually track the position and orientation of the surgical instrument 18 or an implant attached to the surgical instrument 18 during surgery. The at least one electromagnetic field generator 12 may include at least one coil for generating an electromagnetic field. A current is applied from the navigation apparatus 30 to the at least one coil of the at least one electromagnetic field generator 12 to generate a magnetic field around the at least one electromagnetic field generator 12. The at least one electromagnetic sensor 16 may include at least one coil for detecting the magnetic field. The at least one electromagnetic sensor 16 is brought into proximity with the at least one electromagnetic field generator 12 in the surgical field of interest. The magnetic field induces a voltage in the at least one coil of the at least one electromagnetic sensor 16, detecting the magnetic field generated by the at least one electromagnetic field generator 12 for calculating the position and orientation of the at least one surgical instrument 18 or implant. The at least one electromagnetic sensor 16 includes electronics for digitizing magnetic field measurements detected by the at least one electromagnetic sensor 16.

The magnetic field measurements can be used to calculate the position and orientation of the surgical instrument 18 or an implant according to any suitable method or system. After the magnetic field measurements are digitized using electronics, the digitized signals are transmitted from the at least one electromagnetic sensor 16 to the computer on the navigation apparatus 30 through a navigation interface. The digitized signals may be transmitted from the at least one electromagnetic sensor 16 to the navigation apparatus 30 using wired or wireless communication protocols and interfaces. The digitized signals received by the navigation apparatus 30 represent magnetic field information detected by the at least one electromagnetic sensor 16. The digitized signals are used to calculate position and orientation information of the surgical instrument 18 or implant. The position and orientation information is used to register the location of the surgical instrument 18 or implant to acquired imaging data from the imaging apparatus 20. The position and orientation data is visualized on the display 26, showing in real-time the location of the surgical instrument 18 or implant on pre-acquired or real-time images from the imaging apparatus 20. The acquired imaging data from the imaging apparatus 20 may include CT imaging data, MR imaging data, PET imaging data, ultrasound imaging data, X-ray imaging data, or any other suitable imaging data, as well as any combinations thereof. In addition to the acquired imaging data from various modalities, real-time imaging data from various real-time imaging modalities may also be available.

In an exemplary embodiment, the medical navigation system 10 may be integrated into a single integrated imaging and navigation system with integrated instrumentation and software.

In an exemplary embodiment, the medical navigation system 10 may be an electromagnetic navigation system utilizing electromagnetic navigation technology. However, other tracking or navigation technologies may be utilized as well.

FIG. 2 is an exemplary block diagram of an embodiment of a medical navigation system 210. The medical navigation system 210 is illustrated conceptually as a collection of modules and other components that are included in a navigation apparatus 230, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for position and orientation calculations as well as dedicated processors for imaging operations and visualization operations. As a further option, the modules may be implemented using a hybrid configuration in which certain modular functions are performed using dedicated hardware, while the remaining modular functions are performed using an off-the-shelf computer. In the embodiment shown in FIG. 2, the medical navigation system 210 includes a single computer 232 having a processor 234, a system controller 236 and memory 238. The operations of the modules and other components of the navigation apparatus 230 may be controlled by the system controller 236.

The medical navigation system 210 includes at least one electromagnetic field generator 212 that is coupled to a navigation interface 240. The at least one electromagnetic field generator 212 generates at least one electromagnetic field that is detected by at least one electromagnetic sensor 216. The navigation interface 240 receives digitized signals from at least one electromagnetic sensor 216. The navigation interface 240 includes at least one Ethernet port. The at least one Ethernet port may be provided, for example, with an Ethernet network interface card or adapter. However, according to various alternate embodiments, the digitized signals may be transmitted from the at least one electromagnetic sensor 216 to the navigation interface 240 using alternative wired or wireless communication protocols and interfaces.

The digitized signals received by the navigation interface 240 represent magnetic field information from the at least one electromagnetic field generator 212 detected by the at least one electromagnetic sensor 216. In the embodiment illustrated in FIG. 2, the navigation interface 240 transmits the digitized signals to a tracker module 250 over a local interface 242. The tracker module 250 calculates position and orientation information based on the received digitized signals. This position and orientation information provides a location of a surgical instrument or implant.

In an exemplary embodiment, the at least one electromagnetic field generator 212 and the at least one electromagnetic sensor 216 may be coupled to the navigation interface 240 through either a wired or wireless connection.

The tracker module 250 communicates the position and orientation information to a navigation module 260 over a local interface 242. As an example, this local interface 242 is a Peripheral Component Interconnect (PCI) bus. However, according to various alternate embodiments, equivalent bus technologies may be substituted.

Upon receiving the position and orientation information, the navigation module 260 is used to register the location of the surgical instrument or implant to acquired patient data. In the embodiment illustrated in FIG. 2, the acquired patient data is stored on a disk 244. The acquired patient data may include CT data, MR data, PET data, ultrasound data, X-ray data, or any other suitable data, as well as any combinations thereof. By way of example only, the disk 244 is a hard disk drive, but other suitable storage devices may be used.

Patient imaging data acquired prior to the procedure may be transferred to the navigation system and stored on a disk 244. The acquired patient data is loaded into memory 238 from the disk 244. The acquired patient data is retrieved from the disk 244 by a disk controller 246. The navigation module 260 reads from memory 238 the acquired patient data. The navigation module 260 registers the location of the surgical instrument or implant to acquired patient data, and generates image data suitable to visualize the patient image data and a representation of the surgical instrument or implant. The image data is transmitted to a display controller 248 over a local interface 242. The display controller 248 is used to output the image data to display 226.

The medical navigation system 210 may further include an imaging apparatus 220 coupled to an imaging interface 270 for receiving real-time imaging data. The imaging data is processed in an imaging module 280. The imaging apparatus 220 provides the ability to display real-time imaging data in combination with position and orientation information of a surgical instrument or implant on the display 226.

Coupled to display 226 is a user interface 228. The user interface 228 is used to manipulate the registration image displayed on display 226. The user interface 228 may be implemented through standard input tools such as a mouse, keyboard, joystick, pushbuttons, touch screen display, etc.

While one display 226 is illustrated in the embodiment in FIG. 2, alternate embodiments may include various display configurations. Various display configurations may be used to improve operating room ergonomics, display different views, or display information to personnel at various locations.

Generally, image-guided surgery systems operate with an image display which is positioned in a surgeon's field of view and which displays a few panels such as a selected 3D image and several 2D or 3D X-ray or fluoroscopic views taken from different angles. The 3D images typically have a spatial resolution that is both rectilinear and accurate to within a very small tolerance. By contrast, X-ray or fluoroscopic views may be distorted. The X-ray or fluoroscopic views are shadow graphic in that they represent the density of all tissue through which the X-ray beam has passed. In a medical navigation systems, the display visible to the surgeon may show a graphic or CAD representation of a surgical instrument, implant, or other device projected onto an X-ray or fluoroscopic image, so that the surgeon may visualize the position and orientation of the surgical instrument, implant or other device in relation to the imaged patient anatomy.

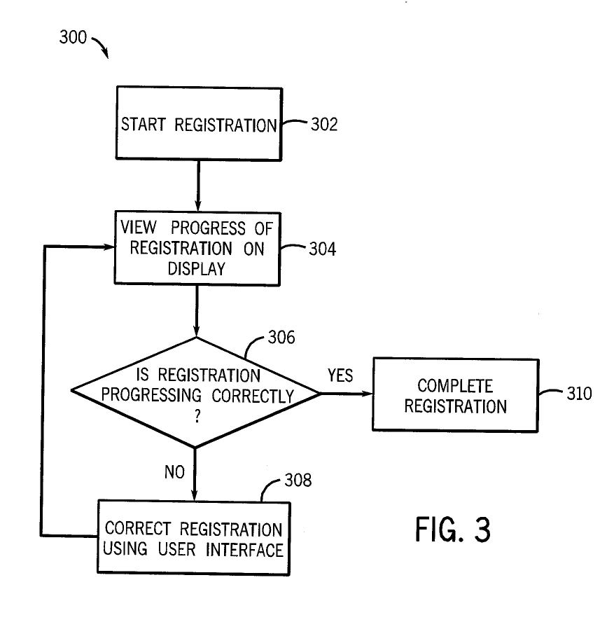

FIG. 3 is an exemplary flow diagram of an embodiment of a method 300 for performing image registration. The method 300 begins at step 302 by performing an initial registration of a second image to a dataset from a first image. The first image may be acquired by a first imaging apparatus. The second image may be acquired by a second imaging apparatus. The first and second imaging apparatus may or may not be the same. The initial registration may be determined by a registration component of a medical navigation system. The initial registration may be based on two or more images. The registration component may be an iterative registration component, for example, adapted to register a sequence of images acquired after a first image.

The registration component uses an image registration algorithm to register a pre-operative 3D image dataset to one or more intra-operative 2D or 3D images. The imaging registration algorithm is iterative and may be started and reset and paused at arbitrary points. The image registration algorithm may also include a feedback mechanism for user interaction. The feedback mechanism is via a direct view of the images and data. This is the presentation with which users of medical navigation systems are familiar.

The dataset may be based at least in part on one or more 3D images. The dataset may be a CT dataset, MR dataset, PET dataset, or an ultrasound dataset. The dataset may be based on a series of image slices of a region of a patient's body. The dataset may include multiple image sets, such as CT, MR, PET, or ultrasound image sets. The image sets may be registered based on fiducials and/or tracking markers.

At step 304, the user may be presented with a live visualization of the registration as it is occurring on a display of the medical navigation system. At step 306, the user may determine if the registration is progressing correctly. For example, the user may be requested to verify that the alignment of the at least two images appears correct in at least one displayed orientation. If there are no misalignments between images, then the registration is completed at step 310. If there are misalignments between images, then the user is provided an opportunity to assist the registration process by manipulating or correcting any misalignments in the registration observed by the user on the display through a user interface at step 308. As the registration is happening, the user is able to manipulate the visualization of the registration to guide the automated registration to a better alignment. The medical navigation system allows the visualization of the registration to be manipulated by the user using a user interface having standard input tools such as a mouse, keyboard, joystick, pushbuttons, touch screen display, etc. This iteration continues until the user is happy with the registration and the registration is completed at step 310.

Subsequent images may be acquired after the second image during the procedure. These images may be a 2D or 3D X-ray or fluoroscopic images. These images may be acquired by an imaging apparatus of the medical navigation system.

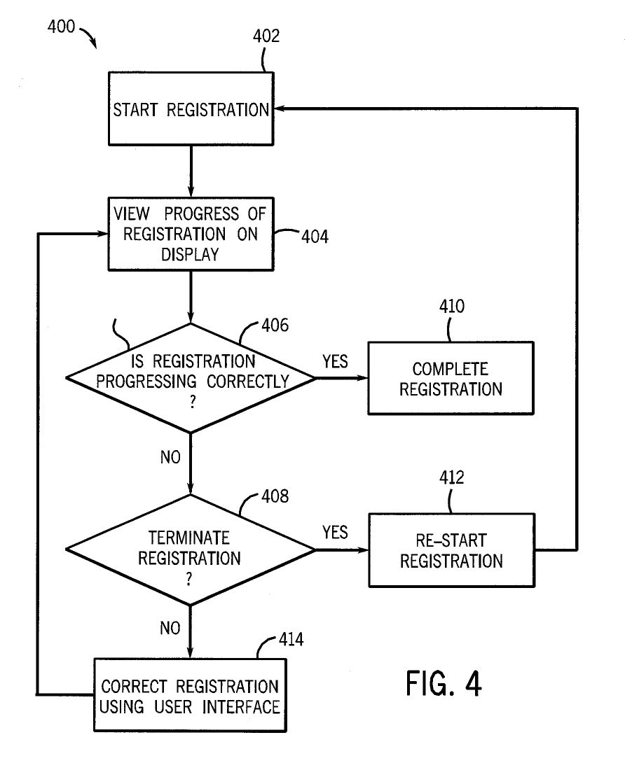

FIG. 4 is an exemplary flow diagram of an embodiment of a method 400 for performing image registration. The method 400 begins at step 402 by performing an initial registration of a second image to a dataset from a first image. The first image may be acquired by a first imaging apparatus. The second image may be acquired by a second imaging apparatus. The first and second imaging apparatus may or may not be the same. The initial registration may be determined by a registration component of a medical navigation system. The initial registration may be based on two or more images. The registration component may be an iterative registration component, for example, adapted to register a sequence of images acquired after a first image.

The registration component uses an image registration algorithm to register a pre-operative 3D image dataset to one or more intra-operative 2D or 3D images. The imaging registration algorithm is iterative and may be started and reset and paused at arbitrary points. The image registration algorithm may also include a feedback mechanism for user interaction. The feedback mechanism is via a direct view of the images and data. This is the presentation with which users of medical navigation systems are familiar.

The dataset may be based at least in part on one or more 3D images. The dataset may be a CT dataset, MR dataset, PET dataset, or an ultrasound dataset. The dataset may be based on a series of image slices of a region of a patient's body. The dataset may include multiple image sets, such as CT, MR, PET, or ultrasound image sets. The image sets may be registered based on fiducials and/or tracking markers.

At step 404, the user may be presented with a live visualization of the registration as it is occurring on a display of the medical navigation system. At step 406, the user may determine if the registration is progressing correctly. For example, the user may be requested to verify that the alignment of the at least two images appears correct in at least one displayed orientation. If there are no misalignments between images, then the registration is completed at step 410. If there are misalignments between images, then the user has the option of terminating the current registration at step 408 or correcting any misalignments in the registration observed by the user on the display through a user interface at step 414. If the user decides to terminate the registration at step 408, the user may re-start the registration at step 412 using a different set of images. This iteration continues until the user accepts the registration and the registration is completed at step 410. At step 414, as the registration is occurring, the user is able to manipulate the visualization of the registration to guide the automated registration to a better alignment. The medical navigation system allows the visualization of the registration to be manipulated by the user using a user interface having standard input tools such as a mouse, keyboard, joystick, pushbuttons, touch screen display, etc. This iteration continues until the user accepts the registration and the registration is completed at step 410.

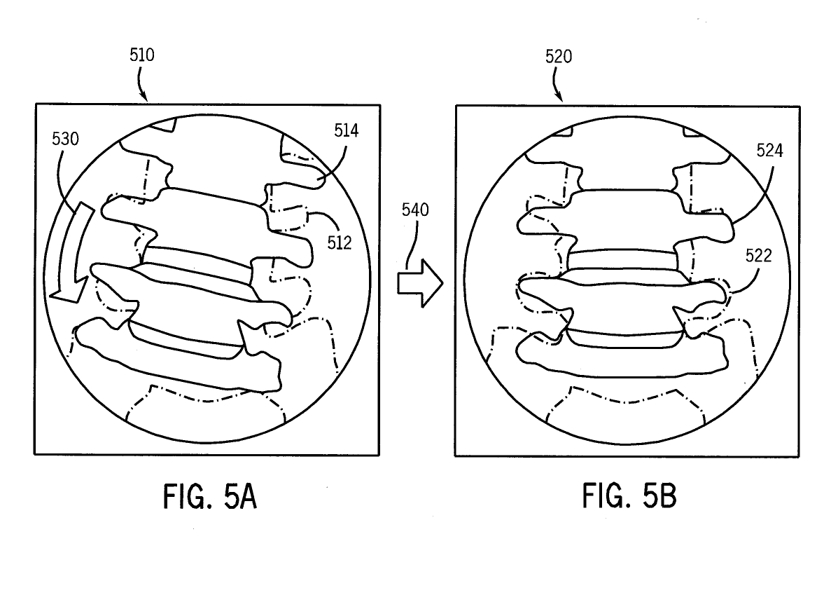

As an example of user interaction, FIG. 5A is an exemplary diagram of a visualization of a misaligned registration 510 on a display of the medical navigation system. FIG. 5B is an exemplary diagram of a visualization of an aligned registration 520 on a display of the medical navigation system after user interaction to correct the visible misalignment in FIG. 5A. In FIG. 5A, a first image 514 is shown misaligned with respect to a second image 512. A graphical representation of an arrow 530 is provided on the display in order to aid the user in manipulating the visualization of the registration for proper alignment as shown in FIG. 5B. To correct the misalignment, the first image 514 is rotated counterclockwise according to arrow 530 to align it with the second image 512. The resulting visualization of the aligned registration 520 as shown in FIG. 5B illustrates the first image 524 now properly aligned with respect to the second image 522 as presented by the graphical representation of arrow 540.

In an exemplary embodiment, the images shown in FIGS. 5A and 5B may be obtained using CT, MR, PET, ultrasound, X-ray or any suitable imaging technology, as well as any combinations thereof.

The methods 300 and 400 are described with reference to elements of systems described above, but it should be understood that other implementations are possible. Certain embodiments may omit one or more of these steps and/or perform the steps in a different order than the order listed. For example, some steps may not be performed in certain embodiments, or certain steps may be performed in a different order, including simultaneously, than listed above.

The methods 300 and 400 described above allow a user to determine how much interaction to provide. If there is a misalignment in the registration, the user may correct the misalignment or choose to try another set of images. If the user is busy during the procedure, the user can let the automated routine work without assistance.

Most registration algorithms try to achieve complete automation. The system and method of this disclosure serve as assistance to qualified and responsible users for their ability to monitor and manipulate the registration process in order to achieve the best registration. It provides an inherent robustness of having a human directly involved in the registration process.

It should be appreciated that according to alternate embodiments, the at least one electromagnetic sensor may be an electromagnetic receiver, an electromagnetic field generator (transmitter), or any combination thereof. Likewise, it should be appreciated that according to alternate embodiments, the at least one electromagnetic field generator may be an electromagnetic receiver, an electromagnetic transmitter or any combination of an electromagnetic field generator (transmitter) and an electromagnetic receiver.

Several embodiments are described above with reference to drawings. These drawings illustrate certain details of specific embodiments that implement the systems, methods and programs of the invention. However, the drawings should not be construed as imposing on the invention any limitations associated with features shown in the drawings. This disclosure contemplates methods, systems and program products on any machine-readable media for accomplishing its operations. As noted above, the embodiments of the may be implemented using an existing computer processor, or by a special purpose computer processor incorporated for this or another purpose or by a hardwired system.

As noted above, embodiments within the scope of the included program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments are described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The foregoing description of embodiments has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

Those skilled in the art will appreciate that the embodiments disclosed herein may be applied to the formation of any medical navigation system. Certain features of the embodiments of the claimed subject matter have been illustrated as described herein, however, many modifications, substitutions, changes and equivalents will now occur to those skilled in the art. Additionally, while several functional blocks and relations between them have been described in detail, it is contemplated by those of skill in the art that several of the operations may be performed without the use of the others, or additional functions or relationships between functions may be established and still be in accordance with the claimed subject matter. It is, therefore, to be understood that the appended claims are intended to cover all such modifications and changes as fall within the true spirit of the embodiments of the claimed subject matter.

While the invention has been described with reference to various embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A medical navigation system comprising:
at least one imaging apparatus adapted to acquire a first image and a second image of a region of interest of a subject;
a registration component adapted to perform a registration of the second image to a dataset of the first image;
at least one display for displaying a visualization of the registration of the second image to a dataset of the first image as it is occurring; and
a user interface for manipulating the visualization of the registration to correct any misalignments between the first image and the second image in the registration.

2. The system of claim 1, wherein the first image is acquired by a first imaging apparatus.

3. The system of claim 1, wherein the first image is acquired prior to a medical procedure and transferred and stored on a storage device of the medical navigation system.

4. The system of claim 3, wherein the second image is acquired by a second imaging apparatus.

5. The system of claim 4, wherein the second image is acquired during the medical procedure.

6. The system of claim 1, wherein the registration component includes a feedback mechanism for user interaction with the registration process.

7. The system of claim 6, wherein the feedback mechanism provides visualization of the second image and the dataset of the first image on the display.

8. The system of claim 1, wherein the acquired first image data is selected from the group consisting of computed tomography data, magnetic resonance data, positron emission tomography data, ultrasound data, and X-ray data and any combinations thereof.

9. The system of claim 1, wherein the acquired second image data is selected from the group consisting of computed tomography data, magnetic resonance data, positron emission tomography data, ultrasound data, and X-ray data and any combinations thereof.

10. The system of claim 1, wherein the at least one display is a touch screen display with graphical inputs.

11. The system of claim 1, wherein the user interface includes standard input tools selected from a group consisting of a mouse, a keyboard, a joystick, a plurality of pushbuttons, and a touch screen display.

12. A method for performing image registration comprising:
acquiring a first image and a second image of a region of interest of a patient;
performing a registration of the second image to a dataset of the first image;
viewing a visualization of the registration on at least one display as the registration is occurring; and
manipulating the visualization of the registration to correct any misalignments between the first image and the second image in the registration using a user interface.

13. The method of claim 12, further comprising the step of terminating the registration and re-starting the registration with a new set of images.

14. The method of claim 12, wherein the first image is acquired prior to a medical procedure and transferred and stored on a storage device of the medical navigation system.

15. The method of claim 14, wherein the second image is acquired during the medical procedure.

16. The method of claim 12, wherein the registration includes a feedback mechanism for user interaction with the registration process.

17. The method of claim 16, wherein the feedback mechanism provides visualization of the second image and the dataset of the first image on the display.

18. The method of claim 12, wherein the at least one display is a touch screen display with graphical inputs.

19. The method of claim 12, wherein the user interface includes standard input tools selected from a group consisting of a mouse, a keyboard, a joystick, a plurality of pushbuttons, and a touch screen display.

20. A computer-readable medium including a set of instructions for execution on a computer, the set of instructions comprising:
an acquisition routine for acquiring a first image and a second image of a region of interest of a patient;
a registration routine for registering the second image to a dataset of the first image;
a visualization routine for visualizing the registration on a display while the registration is proceeding; and
a user interaction routine for manipulating the registration to correct any misalignments between the first image and the second image.
